(19) United States
(12) Patent Application Publication
(10) Pub. No.: US 2023/0233435 A1
(43) Pub. Date: Jul. 27, 2023

(54) COSMETIC COMPOSITION, COSMETIC, AND METHOD FOR APPLYING COSMETIC COMPOSITION

(71) Applicant: UNDER7 Co., Ltd., Tokyo (JP)

(72) Inventor: Keita MINAMI, Tokyo (JP)

(21) Appl. No.: 18/080,389

(22) Filed: Dec. 13, 2022

(30) Foreign Application Priority Data
Jan. 21, 2022 (JP) 2022-007964
Jun. 7, 2022 (JP) 2022-092500

(51) Int. Cl. A61K 8/60; A61K 8/362; A61K 8/44; A61K 8/365; A61Q 5/00; A61Q 19/00

(52) U.S. Cl. CPC A61K 8/606; A61K 8/362; A61K 8/44; A61K 8/365; A61Q 5/00; A61Q 19/00; A61K 8/60; A61K 2800/5922

(57) ABSTRACT

A cosmetic composition exerts a high improving effect on a texture of hair of a head or a scalp. The cosmetic composition includes nucleic acid, succinic acid, alanine, citric acid, and glycine. An amount of the nucleic acid is 6.6 to 11.5% by mass based on a total mass of the composition, and an amount of the citric acid is 7.1 to 17.4% by mass based on the total mass of the composition. The cosmetic composition is allowed to contain at least one member selected from, for example, histidine, arginine, proline, isoleucine, serine, valine, aspartic acid, threonine, phenylalanine, pyrrolidonecarboxylic acid, sodium pyrrolidonecarboxylate, sodium lactate, and water to prepare a cosmetic.

### CROSS-REFERENCE TO RELATED APPLICATION

The present application is based on and claims priorities from Japanese Patent Application No. 2022-007964 filed on Jan. 21, 2022 and Japanese Patent Application No. 2022-092500 filed on Jun. 7, 2022, the disclosure of which is hereby incorporated by reference herein in its entirety.

### BACKGROUND OF THE INVENTION

### Field of the Invention

The present invention relates to a cosmetic composition, a cosmetic comprising this cosmetic composition, and a method for applying a cosmetic composition.

### Description of the Related Art

Cosmetic compositions supplemented with various substances have heretofore been known for the purpose of imparting specific effects to cosmetics. For example, cosmetic compositions supplemented with nucleic acid or citric acid are known in the art in order to improve the texture of hair of the head or the scalp.

For example, there exists Japanese Patent Laid-Open No. 2018-203628 as a prior art document that discloses this kind of cosmetic composition. The invention disclosed in Japanese Patent Laid-Open No. 2018-203628 can reportedly provide a cosmetic composition or a food or drink composition containing a substance that provides resilience or gloss to the skin or hair of the head and offers very favorable touch or usability of the skin or hair of the head, such as an improved moisturizing effect, whitening effect, or combability of hair, when the composition is used externally or internally.

However, conventional techniques typified by Japanese Patent Laid-Open No. 2018-203628 described above, in many cases, cannot sufficiently exert their effects even if cosmetics are supplemented with components such as nucleic acid or citric acid. The conventional techniques have found a solution by an approach such as increase in the amount of nucleic acid, citric acid, or the like added and however, present problems associated with cost and stability in formulations. Particularly, for improvement in the texture of hair of the head or the scalp, it is necessary to optimize substance components constituting a cosmetic composition. Thus, there has been a demand for the achievement of a novel cosmetic composition that exerts a high improving effect.

The present invention has been made in light of the problems with the conventional techniques mentioned above.

An object of the present invention is to provide a cosmetic composition having a novel formulation of components that can exert a high improving effect on the texture of hair of the head or the scalp, a cosmetic comprising this cosmetic composition, and a method for applying a cosmetic composition.

### SUMMARY OF THE INVENTION

The cosmetic composition according to the present invention comprises

(a) nucleic acid,
(b) succinic acid,
(c) alanine,
(d) citric acid, and
(e) glycine, wherein
an amount of the nucleic acid (a) is 6.6 to 11.5% by mass based on the total mass of the composition, and
an amount of the citric acid (d) is 7.1 to 17.4% by mass based on the total mass of the composition.

The cosmetic composition according to the present invention can be configured such that

an amount of the succinic acid (b) is 3.6 to 11.5% by mass based on the total mass of the composition,
an amount of the alanine (c) is 3.6 to 11.5% by mass based on the total mass of the composition, and
an amount of the glycine (e) is 3.6 to 23.0% by mass based on the total mass of the composition.

The cosmetic composition according to the present invention can further comprise

(f) tartaric acid, wherein
the amount of the nucleic acid (a) is 6.6 to 6.7% by mass based on the total mass of the composition,
the amount of the succinic acid (b) is 6.6 to 6.7% by mass based on the total mass of the composition,
the amount of the alanine (c) is 6.6 to 6.7% by mass based on the total mass of the composition,
the amount of the citric acid (d) is 10.0% by mass based on the total mass of the composition,
the amount of the glycine (e) is 13.3 to 13.4% by mass based on the total mass of the composition, and
an amount of the tartaric acid (f) is 56.6 to 56.8% by mass based on the total mass of the composition.

The cosmetic composition according to the present invention can further comprise

(f) tartaric acid, and
(g) arginine, wherein
the amount of the nucleic acid (a) is 10.0 to 11.5% by mass based on the total mass of the composition,
the amount of the succinic acid (b) is 10.0 to 11.5% by mass based on the total mass of the composition,
the amount of the alanine (c) is 10.0 to 11.5% by mass based on the total mass of the composition,
the amount of the citric acid (d) is 15.0 to 17.4% by mass based on the total mass of the composition,
the amount of the glycine (e) is 20.0 to 23.0% by mass based on the total mass of the composition,
an amount of the tartaric acid (f) is 2.5 to 30.0% by mass based on the total mass of the composition, and
an amount of the arginine (g) is 5.0 to 23.0% by mass based on the total mass of the composition.

The cosmetic composition according to the present invention can be configured such that

the amount of the nucleic acid (a) is 10.0 to 10.3% by mass based on the total mass of the composition,
the amount of the succinic acid (b) is 10.0 to 10.3% by mass based on the total mass of the composition,
the amount of the alanine (c) is 10.0 to 10.3% by mass based on the total mass of the composition,
the amount of the citric acid (d) is 15.0 to 15.4% by mass based on the total mass of the composition,
the amount of the glycine (e) is 20.0 to 20.6% by mass based on the total mass of the composition,
the amount of the tartaric acid (f) is 28.1 to 30.0% by mass based on the total mass of the composition, and
the amount of the arginine (g) is 5.0 to 5.2% by mass based on the total mass of the composition.

The cosmetic composition according to the present invention can be configured such that

the amount of the nucleic acid (a) is 11.4 to 11.5% by mass based on the total mass of the composition,
the amount of the succinic acid (b) is 11.4 to 11.5% by mass based on the total mass of the composition,
the amount of the alanine (c) is 11.4 to 11.5% by mass based on the total mass of the composition,
the amount of the citric acid (d) is 17.2 to 17.4% by mass based on the total mass of the composition,
the amount of the glycine (e) is 23.0% by mass based on the total mass of the composition,
the amount of the tartaric acid (f) is 10.3 to 10.4% by mass based on the total mass of the composition, and
the amount of the arginine (g) is 14.9 to 15.0% by mass based on the total mass of the composition.

The cosmetic composition according to the present invention can be configured such that

the amount of the nucleic acid (a) is 11.3 to 11.5% by mass based on the total mass of the composition,
the amount of the succinic acid (b) is 11.3 to 11.5% by mass based on the total mass of the composition,
the amount of the alanine (c) is 11.3 to 11.5% by mass based on the total mass of the composition,
the amount of the citric acid (d) is 17.0 to 17.2% by mass based on the total mass of the composition,
the amount of the glycine (e) is 22.6 to 23.0% by mass based on the total mass of the composition,
the amount of the tartaric acid (f) is 2.5 to 3.4% by mass based on the total mass of the composition, and
the amount of the arginine (g) is 22.7 to 23.0% by mass based on the total mass of the composition.

The cosmetic composition according to the present invention can further comprise

(g) arginine, and
(h) trehalose, wherein
the amount of the nucleic acid (a) is 7.0 to 7.2% by mass based on the total mass of the composition,
the amount of the succinic acid (b) is 3.6% by mass based on the total mass of the composition,
the amount of the alanine (c) is 3.6% by mass based on the total mass of the composition,
the amount of the citric acid (d) is 7.1 to 7.2% by mass based on the total mass of the composition,
the amount of the glycine (e) is 3.6% by mass based on the total mass of the composition,
an amount of the arginine (g) is 71.4 to 71.5% by mass based on the total mass of the composition, and
an amount of the trehalose (h) is 3.6% by mass based on the total mass of the composition.

Another cosmetic composition according to the present invention comprises

(e) glycine, and
(h) trehalose, wherein
an amount of the glycine (e) is 50.0% by mass based on the total mass of the composition, and
an amount of the trehalose (h) is 50.0% by mass based on the total mass of the composition.

The cosmetic according to the present invention can be formed by incorporating one or more members selected from histidine, arginine, proline, isoleucine, serine, valine, aspartic acid, threonine, phenylalanine, pyrrolidonecarboxylic acid, sodium pyrrolidonecarboxylate, sodium lactate, and water in the cosmetic composition.

The cosmetic according to the present invention can be formed by incorporating one or more members selected from histidine, proline, isoleucine, serine, valine, aspartic acid, threonine, phenylalanine, pyrrolidonecarboxylic acid, sodium pyrrolidonecarboxylate, sodium lactate, and water in the cosmetic composition.

The cosmetic according to the present invention can be formed by incorporating one or more members selected from histidine, proline, isoleucine, serine, valine, aspartic acid, threonine, phenylalanine, pyrrolidonecarboxylic acid, sodium pyrrolidonecarboxylate, sodium lactate, and water in the cosmetic composition.

The cosmetic according to the present invention can be formed by incorporating one or more members selected from histidine, proline, isoleucine, serine, valine, aspartic acid, threonine, phenylalanine, pyrrolidonecarboxylic acid, sodium pyrrolidonecarboxylate, sodium lactate, and water in the cosmetic composition.

The cosmetic according to the present invention can be formed by incorporating one or more members selected from histidine, proline, isoleucine, serine, valine, aspartic acid, threonine, phenylalanine, pyrrolidonecarboxylic acid, sodium pyrrolidonecarboxylate, sodium lactate, and water in the cosmetic composition.

The cosmetic according to the present invention can be formed by incorporating one or more members selected from arginine, alanine, histidine, proline, isoleucine, serine, valine, aspartic acid, threonine, phenylalanine, pyrrolidonecarboxylic acid, sodium pyrrolidonecarboxylate, sodium lactate, and water in the cosmetic composition.

The present invention includes a method for applying a cosmetic composition, the method comprising applying the cosmetic composition described above to hair, the skin, hair of the head and/or the scalp.

The present invention can provide a cosmetic composition having a novel formulation of components that can exert a high improving effect on the texture of hair of the head or the scalp, a cosmetic comprising this cosmetic composition, and a method for applying a cosmetic composition.

### BRIEF DESCRIPTION OF THE DRAWINGS

FIG. 1 is a diagram showing Embodiments 1 to 5 which are exemplary embodiments that can be adopted by the cosmetic composition according to the present invention;

FIG. 2 is a diagram showing Embodiment 6 which is another exemplary embodiment that can be adopted by the cosmetic composition according to the present invention;

FIG. 3 is a diagram showing Examples 1-1 to 1-4 which are specific examples of the cosmetic composition according to Embodiment 1 shown in FIG. 1;

FIG. 4 is a diagram showing Examples 2-1 to 2-7 which are specific examples of the cosmetic composition according to Embodiment 2 shown in FIG. 1;

FIG. 5 is a diagram showing Examples 3-1 to 3-4 which are specific examples of the cosmetic composition according to Embodiment 3 shown in FIG. 1;

FIG. 6 is a diagram showing Examples 4-1 to 4-7 which are specific examples of the cosmetic composition according to Embodiment 4 shown in FIG. 1;

FIG. 7 is a diagram showing Examples 5-1 to 5-4 which are specific examples of the cosmetic composition according to Embodiment 5 shown in FIG. 1; and

FIG. 8 is a diagram showing Examples 6-1 to 6-4 which are specific examples of the cosmetic composition according to Embodiment 6 shown in FIG. 2.

### DETAILED DESCRIPTION OF THE PREFERRED EMBODIMENTS

The present inventor has conducted diligent studies to find a cosmetic composition having a novel formulation of components that can exert a high improving effect on the texture of hair of the head, the scalp, or the like by examining various components and applying them to hair, the skin, hair of the head and/or the scalp. As a result, the present inventor has completed the present invention by finding that heretofore known components such as nucleic acid and citric acid as well as components such as succinic acid, alanine, glycine, tartaric acid, arginine, and trehalose are contained while the amounts of these substance components added are optimized, whereby the resulting cosmetic composition provides resilience or gloss to hair of the head, the scalp, hair, or the skin and is capable of exerting an effect such as improvement in hair cuticle or improvement in moisturizing effect, whitening effect, or combability of hair.

Hereinafter, the preferred embodiments of the present invention will be described with reference to the drawings. The following embodiments do not limit the invention, and all combinations of features described in the embodiments are not always essential for the solution of the invention.

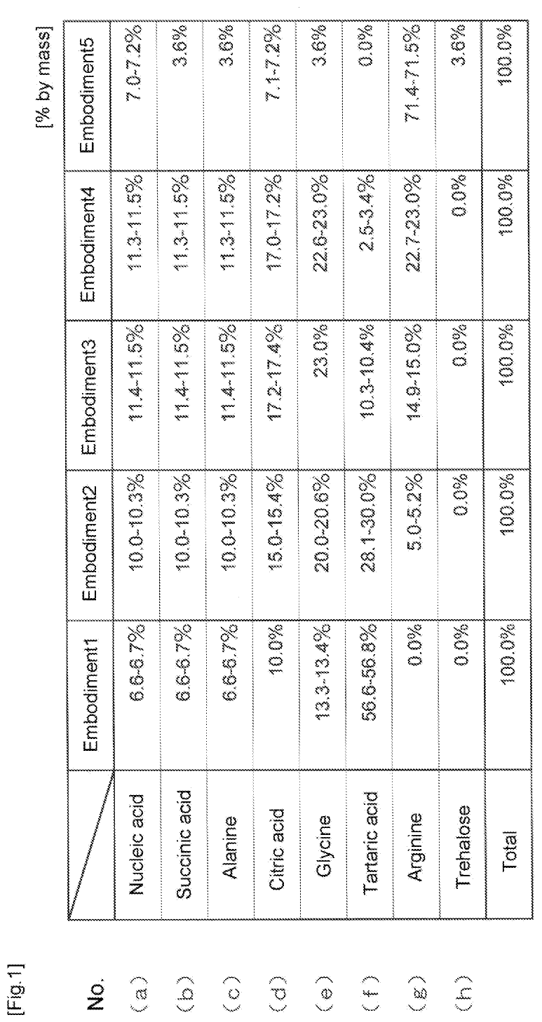

FIG. 1 is a diagram showing Embodiments 1 to 5, which are exemplary embodiments that can be adopted by the cosmetic composition according to the present invention. Hereinafter, each embodiment will be described with reference to FIG. 1.

### Embodiment 1

The cosmetic composition of Embodiment 1 comprises

(a) nucleic acid,
(b) succinic acid,
(c) alanine,
(d) citric acid,
(e) glycine, and
(f) tartaric acid, wherein
the amount of the nucleic acid (a) is 6.6 to 6.7% by mass based on the total mass of the composition,
the amount of the succinic acid (b) is 6.6 to 6.7% by mass based on the total mass of the composition,
the amount of the alanine (c) is 6.6 to 6.7% by mass based on the total mass of the composition,
the amount of the citric acid (d) is 10.0% by mass based on the total mass of the composition,
the amount of the glycine (e) is 13.3 to 13.4% by mass based on the total mass of the composition, and
an amount of the tartaric acid (f) is 56.6 to 56.8% by mass based on the total mass of the composition.

The nucleic acid (a) is a substance that resides in cells that are basic units to form the human body, and is an essential component for producing new cells. For example, in hair, the nucleic acid (a) functions as a component that activates cells of hair and regenerates hair. In short, the nucleic acid (a) can be regarded as a substance indispensable for forming hair.

The succinic acid (b) is a dicarboxylic acid and is a substance having a function of protecting fibrils of hair. The succinic acid (b) also functions as a substance that adjusts pH.

Each of the alanine (c) and the glycine (e) is a non-essential amino acid and is a substance necessary for repairing hair and thereby softening hair or imparting soft finish to the texture of hair. The alanine (c) and the glycine (e) each have the effect of cross-linking amino acids and can produce the effect of strengthening hair, i.e., enhancing the bearing force of hair to render the hair firm.

The citric acid (d) is a tricarboxylic acid and can exert the effect of protecting hair so as not to damage fibrils of hair. The citric acid (d) exerts a chelating effect or fulfils a function as a sequestering agent and the citric acid (d) can therefore also produce the effect of stabilizing the cosmetic composition. The citric acid (d) further functions as a substance that adjusts pH. Trisodium citrate is used as the citric acid of Embodiment 1, and different citric acid, i.e., monosodium citrate or disodium citrate, may be used. In short, the citric acid in Embodiment 1 can include monosodium citrate, disodium citrate or trisodium citrate.

The tartaric acid (f) is a dicarboxylic acid and can fulfil a function of protecting hair through adsorption to fibrils of hair. The tartaric acid (f) also functions as a substance that exerts the effect of lowering a pH value.

The cosmetic composition of Embodiment 1 has the substances having the characteristics mentioned above at the component blending ratio described above and can thereby exert a high improving effect on the texture of hair of the head or the scalp. Particularly, the cosmetic composition of Embodiment 1 has the effect of largely improving the effect of a dicarboxylic acid, i.e., the effect of protecting hair through adsorption to fibrils of hair, because the amount of the tartaric acid (f) is set to as high as 56.6 to 56.8% by mass based on the total mass of the composition. The cosmetic composition of Embodiment 1 also has a sterilizing effect or a peeling effect on hair or the skin because its pH value is acidic pH of 2.5 to 3.0. This cosmetic composition can produce, for example, the effect of softening and lysing old dead cells on the surface of the skin or promoting the metabolism of the body.

### Embodiments 2 to 4

The cosmetic composition of any of Embodiments 2 to 4 comprises

(a) nucleic acid,
(b) succinic acid,
(c) alanine,
(d) citric acid,
(e) glycine,
(f) tartaric acid, and
(g) arginine, wherein
the amount of the nucleic acid (a) is 10.0 to 11.5% by mass based on the total mass of the composition,
the amount of the succinic acid (b) is 10.0 to 11.5% by mass based on the total mass of the composition,
the amount of the alanine (c) is 10.0 to 11.5% by mass based on the total mass of the composition,
the amount of the citric acid (d) is 15.0 to 17.4% by mass based on the total mass of the composition,
the amount of the glycine (e) is 20.0 to 23.0% by mass based on the total mass of the composition,
an amount of the tartaric acid (f) is 2.5 to 30.0% by mass based on the total mass of the composition, and
an amount of the arginine (g) is 5.0 to 23.0% by mass based on the total mass of the composition.

Particularly, the cosmetic composition of Embodiment 2 is configured such that, in addition to the component conditions described above,

the amount of the nucleic acid (a) is 10.0 to 10.3% by mass based on the total mass of the composition,
the amount of the succinic acid (b) is 10.0 to 10.3% by mass based on the total mass of the composition,
the amount of the alanine (c) is 10.0 to 10.3% by mass based on the total mass of the composition,
the amount of the citric acid (d) is 15.0 to 15.4% by mass based on the total mass of the composition,
the amount of the glycine (e) is 20.0 to 20.6% by mass based on the total mass of the composition,
the amount of the tartaric acid (f) is 28.1 to 30.0% by mass based on the total mass of the composition, and
the amount of the arginine (g) is 5.0 to 5.2% by mass based on the total mass of the composition.

Particularly, the cosmetic composition of Embodiment 3 is configured such that, in addition to the component conditions described above,

the amount of the nucleic acid (a) is 11.4 to 11.5% by mass based on the total mass of the composition,
the amount of the succinic acid (b) is 11.4 to 11.5% by mass based on the total mass of the composition,
the amount of the alanine (c) is 11.4 to 11.5% by mass based on the total mass of the composition,
the amount of the citric acid (d) is 17.2 to 17.4% by mass based on the total mass of the composition,
the amount of the glycine (e) is 23.0% by mass based on the total mass of the composition,
the amount of the tartaric acid (f) is 10.3 to 10.4% by mass based on the total mass of the composition, and
the amount of the arginine (g) is 14.9 to 15.0% by mass based on the total mass of the composition.

Particularly, the cosmetic composition of Embodiment 4 is configured such that, in addition to the component conditions described above,

the amount of the nucleic acid (a) is 11.3 to 11.5% by mass based on the total mass of the composition,
the amount of the succinic acid (b) is 11.3 to 11.5% by mass based on the total mass of the composition,
the amount of the alanine (c) is 11.3 to 11.5% by mass based on the total mass of the composition,
the amount of the citric acid (d) is 17.0 to 17.2% by mass based on the total mass of the composition,
the amount of the glycine (e) is 22.6 to 23.0% by mass based on the total mass of the composition,
the amount of the tartaric acid (f) is 2.5 to 3.4% by mass based on the total mass of the composition, and
the amount of the arginine (g) is 22.7 to 23.0% by mass based on the total mass of the composition.

The nucleic acid (a) is a substance that resides in cells that are basic units to form the human body, and is an essential component for producing new cells. For example, in hair, the nucleic acid (a) functions as a component that activates cells of hair and regenerates hair. In short, the nucleic acid (a) can be regarded as a substance indispensable for forming hair.

The succinic acid (b) is a dicarboxylic acid and is a substance having a function of protecting fibrils of hair. The succinic acid (b) also functions as a substance that adjusts pH.

Each of the alanine (c) and the glycine (e) is a non-essential amino acid and is a substance necessary for repairing hair and thereby softening hair or imparting soft finish to the texture of hair. The alanine (c) and the glycine (e) each have the effect of cross-linking amino acids and can produce the effect of strengthening hair, i.e., enhancing the bearing force of hair to render the hair firm.

The citric acid (d) is a tricarboxylic acid and can exert the effect of protecting hair so as not to damage fibrils of hair. The citric acid (d) exerts a chelating effect or fulfils a function as a sequestering agent and the citric acid (d) can therefore also produce the effect of stabilizing the cosmetic composition. The citric acid (d) further functions as a substance that adjusts pH. Trisodium citrate is used as the citric acid of any of Embodiments 2 to 4, and different citric acid, i.e., monosodium citrate or disodium citrate, may be used. In short, the citric acid in any of Embodiments 2 to 4 can include monosodium citrate, disodium citrate or trisodium citrate.

The tartaric acid (f) is a dicarboxylic acid and can fulfil a function of protecting hair through adsorption to fibrils of hair. The tartaric acid (f) also functions as a substance that exerts the effect of lowering a pH value.

The arginine (g) is an amino acid also contained in hair and is a substance for use as an alkali agent. The arginine (g) is also a substance having the effect of promoting the secretion of growth hormone, the effect of enhancing immune strength, and the effect of detoxicating ammonia. The arginine (g) further has the force to largely open hair cuticle and therefore allows the cosmetic composition of any of Embodiments 2 to 4 to penetrate into hair, where the cosmetic composition can exert the effect of improving the texture of hair.

The cosmetic composition of any of Embodiments 2 to 4 has the substances having the characteristics mentioned above at the component blending ratio described above and can thereby exert a high improving effect on the texture of hair of the head or the scalp.

Particularly, the cosmetic composition of Embodiment 2 has a weakly acidic pH value of 4.0 and is thus a composition having a pH value closest to 4.5 to 5.5, which is a pH value at which hair stabilizes. Among the cosmetic compositions of Embodiments 2 to 4, the cosmetic composition of Embodiment 2 has an enhanced treatment effect and can also produce the effect of alleviating damage on hair, because the amount of the tartaric acid (f) is increased with the amount of the arginine (g) reduced.

Meanwhile, the cosmetic composition of Embodiment 3 has the components adjusted so as to have a neutral pH value on the order of 6.0 to 7.5, and can therefore produce the effect of improved force to protect hair. Specifically, the cosmetic composition of Embodiment 3 can be used for, for example, already damaged hair so that the cosmetic composition of Embodiment 3 protects or strengthens hair through adsorption to fibrils of hair. As a result, the cosmetic composition of Embodiment 3 can produce a hair treatment effect or the effect of improving the force to protect hair.

The cosmetic composition of Embodiment 4 has the components adjusted so as to have a weakly alkaline to alkaline pH value of 7.5 or higher with acidity reduced, because the percentage of the arginine (g) is increased. The cosmetic composition of Embodiment 4 can keep alkalinity without lowering a pH value too much, and thereby prevent damage on hair of the head due to an agent such as a bleach without reducing the power of the agent, for example, when hair of the head is colored or bleached. Therefore, this cosmetic composition enables various procedures to be carried out for hair of the head while the hair of the head is protected. This effect is an effect brought about by elevating the percentages of the arginine (g) as well as the citric acid (d).

### Embodiment 5

The cosmetic composition of Embodiment 5 comprises

(a) nucleic acid,
(b) succinic acid,
(c) alanine,
(d) citric acid,
(e) glycine,
(g) arginine, and
(h) trehalose, wherein
the amount of the nucleic acid (a) is 7.0 to 7.2% by mass based on the total mass of the composition,
the amount of the succinic acid (b) is 3.6% by mass based on the total mass of the composition,
the amount of the alanine (c) is 3.6% by mass based on the total mass of the composition,
the amount of the citric acid (d) is 7.1 to 7.2% by mass based on the total mass of the composition,
the amount of the glycine (e) is 3.6% by mass based on the total mass of the composition,
an amount of the arginine (g) is 71.4 to 71.5% by mass based on the total mass of the composition, and
an amount of the trehalose (h) is 3.6% by mass based on the total mass of the composition.

The nucleic acid (a) is a substance that resides in cells that are basic units to form the human body, and is an essential component for producing new cells. For example, in hair, the nucleic acid (a) functions as a component that activates cells of hair and regenerates hair. In short, the nucleic acid (a) can be regarded as a substance indispensable for forming hair.

The succinic acid (b) is a dicarboxylic acid and is a substance having a function of protecting fibrils of hair. The succinic acid (b) also functions as a substance that adjusts pH.

Each of the alanine (c) and the glycine (e) is a non-essential amino acid and is a substance necessary for repairing hair and thereby softening hair or imparting soft finish to the texture of hair. The alanine (c) and the glycine (e) each have the effect of cross-linking amino acids and can produce the effect of strengthening hair, i.e., enhancing the bearing force of hair to render the hair firm.

The citric acid (d) is a tricarboxylic acid and can exert the effect of protecting hair so as not to damage fibrils of hair. The citric acid (d) exerts a chelating effect or fulfils a function as a sequestering agent and the citric acid (d) can therefore also produce the effect of stabilizing the cosmetic composition. The citric acid (d) further functions as a substance that adjusts pH. Trisodium citrate is used as the citric acid of Embodiment 5, and different citric acid, i.e., monosodium citrate or disodium citrate, may be used. In short, the citric acid in Embodiment 5 can include monosodium citrate, disodium citrate or trisodium citrate.

The arginine (g) is an amino acid also contained in hair and is a substance for use as an alkali agent. The arginine (g) is also a substance having the effect of promoting the secretion of growth hormone, the effect of enhancing immune strength, and the effect of detoxicating ammonia. The arginine (g) further has the force to largely open hair cuticle and therefore allows the cosmetic composition of Embodiment 5 to penetrate into hair, where the cosmetic composition can exert the effect of improving the texture of hair.

The trehalose (h) has the effect of preventing the disruption of proteins contained in hair and can exert the effect of enhancing hair's ability to retain moisture.

The cosmetic composition of Embodiment 5 has the substances having the characteristics mentioned above at the component blending ratio described above and is thereby effectively applied to more damaged hair as compared with the cosmetic compositions of Embodiments 1 to 4 mentioned above. Particularly, the cosmetic composition of Embodiment 5 is promoted to penetrate to the inside of hair, where the cosmetic composition can enhance improvement in the quality of hair, because the contents of the arginine (g) and the trehalose (h) are set to elevated levels.

The cosmetic compositions of Embodiments 1 to 5 mentioned above are appropriately selected depending on the state of hair, the skin, hair of the head and/or the scalp and applied thereto and can thereby exert a high improving effect on the texture of hair of the head, the scalp, or the like.

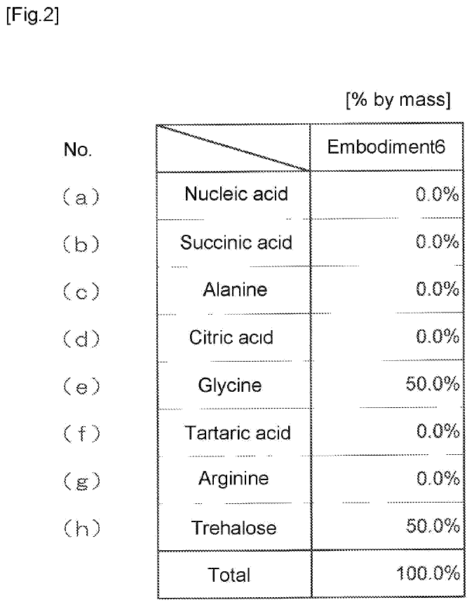

Next, Embodiment 6, which is another exemplary embodiment that can be adopted by the cosmetic composition according to the present invention, will be described with reference to FIG. 2. FIG. 2 is a diagram showing Embodiment 6, which is another exemplary embodiment that can be adopted by the cosmetic composition according to the present invention.

### Embodiment 6

The cosmetic composition of Embodiment 6 comprises

(e) glycine, and
(h) trehalose, wherein
an amount of the glycine (e) is 50.0% by mass based on the total mass of the composition, and
an amount of the trehalose (h) is 50.0% by mass based on the total mass of the composition.

The glycine (e) is a non-essential amino acid and is a substance necessary for repairing hair and thereby softening hair or imparting soft finish to the texture of hair. The glycine (e) has the effect of cross-linking amino acids and can produce the effect of strengthening hair, i.e., enhancing the bearing force of hair to render the hair firm.

The trehalose (h) has the effect of preventing the disruption of proteins contained in hair and can exert the effect of enhancing hair's ability to retain moisture.

The cosmetic composition of Embodiment 6 has the substances having the characteristics mentioned above at the component blending ratio described above and can thereby exert an effect dedicated to the efficacy of the glycine (e) and the trehalose (h) as compared with the cosmetic compositions of Embodiments 1 to 5 mentioned above. Specifically, the cosmetic composition of Embodiment 6 can produce an improving effect on the quality of particularly damaged hair having no bearing force, because the effect of enhancing the bearing force of hair, which is exerted by the glycine (e), is reinforced by the effect of promoting the penetration of the composition to the inside of hair, which is exerted by the trehalose (h).

### Examples 1 to 6

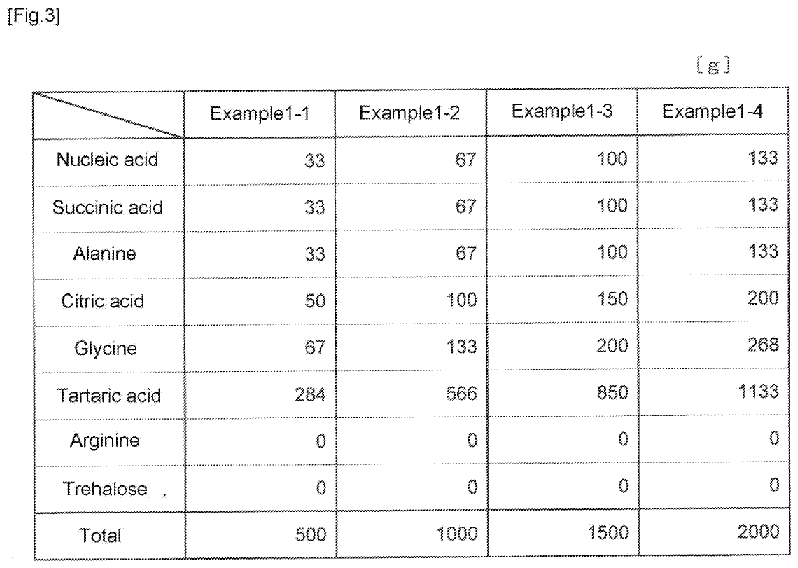

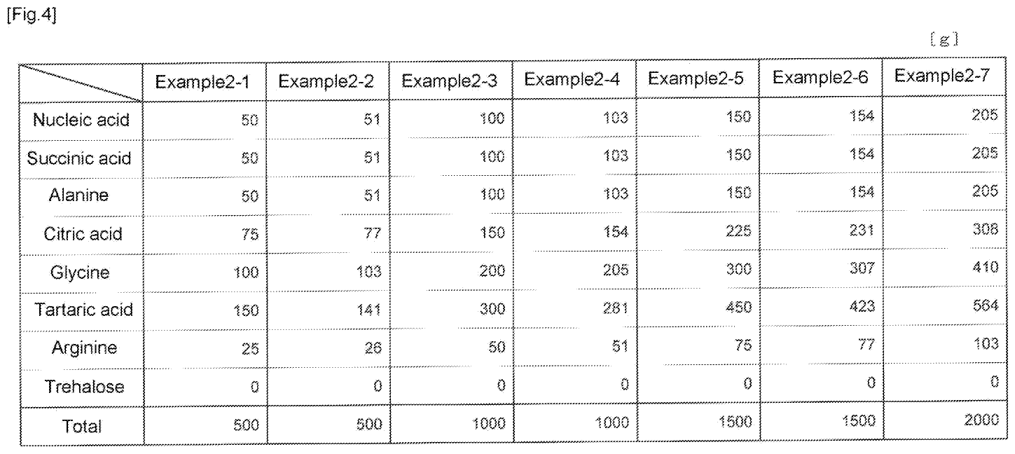

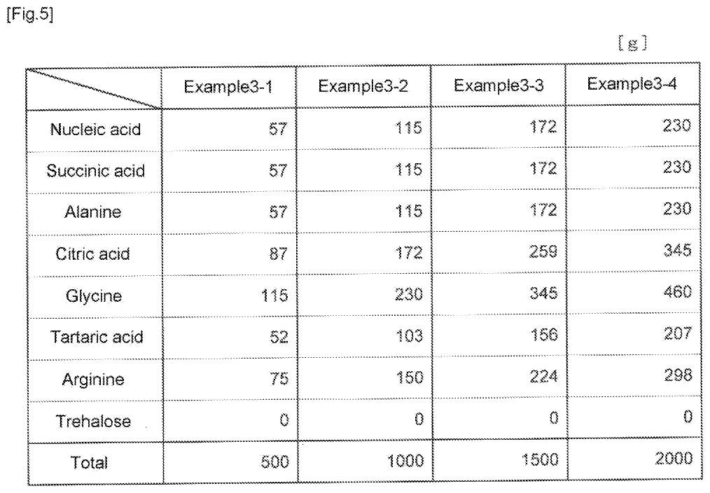

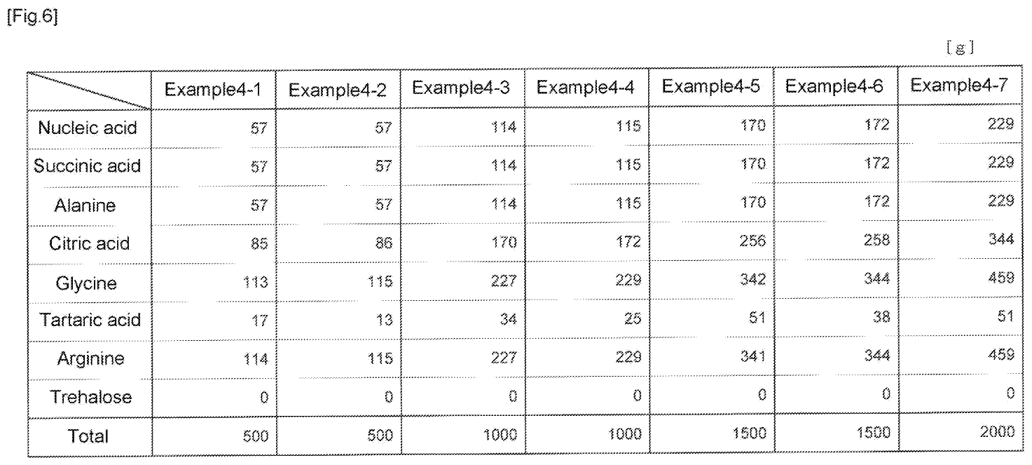

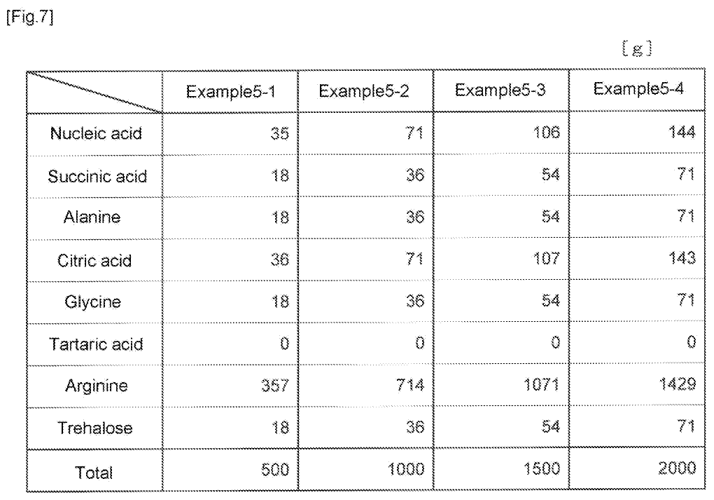

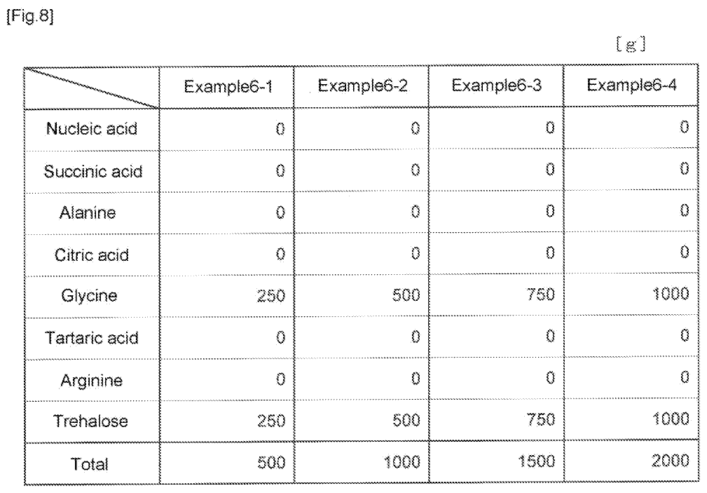

The preferred embodiments of the present invention are described above. Next, specific examples of Embodiments 1 to 6 mentioned above will be given with reference to FIGS. 3 to 8. In this context, FIG. 3 is a diagram showing Examples 1-1 to 1-4, which are specific examples of the cosmetic composition according to Embodiment 1 shown in FIG. 1. FIG. 4 is a diagram showing Examples 2-1 to 2-7, which are specific examples of the cosmetic composition according to Embodiment 2 shown in FIG. 1. FIG. 5 is a diagram showing Examples 3-1 to 3-4, which are specific examples of the cosmetic composition according to Embodiment 3 shown in FIG. 1. FIG. 6 is a diagram showing Examples 4-1 to 4-7, which are specific examples of the cosmetic composition according to Embodiment 4 shown in FIG. 1. FIG. 7 is a diagram showing Examples 5-1 to 5-4, which are specific examples of the cosmetic composition according to Embodiment 5 shown in FIG. 1. FIG. 8 is a diagram showing Examples 6-1 to 6-4, which are specific examples of the cosmetic composition according to Embodiment 6 shown in FIG. 2.

FIGS. 3 to 8 illustrate specific amounts of the components for forming cosmetic compositions having component ratios according to the inventive formulations shown in Embodiments 1 to 6 mentioned above. For example, FIG. 3 indicates a specific quantity of each component by the number of grams as Example 1-1 when 500 g in total of the cosmetic composition according to Embodiment 1 mentioned above is formed. Likewise, Example 1-2 in FIG. 3 depicts a specific quantity of each component when 1000 g in total of the cosmetic composition according to Embodiment 1 mentioned above is formed. Example 1-3 in FIG. 3 depicts a specific quantity of each component when 1500 g in total of the cosmetic composition according to Embodiment 1 mentioned above is formed. Example 1-4 in FIG. 3 depicts a specific quantity of each component when 2000 g in total of the cosmetic composition according to Embodiment 1 mentioned above is formed.

The specific amounts of the components in the cosmetic composition of the present invention will become obvious with reference to Examples 1-1 to 6-4 illustrated in FIGS. 3 to 8. The cosmetic composition according to each example can also exert the working effects described for the embodiment mentioned above, irrespective of the number of grams of the total quantity, as long as the cosmetic composition has any of the component ratios shown in Embodiments 1 to 6 mentioned above.

The preferred embodiments of the present invention are described above. However, the technical scope of the present invention is not limited to the scopes described in the embodiments described above. Various changes or modifications can be made in the embodiments described above.

For example, the cosmetic compositions of Embodiments 1 to 5 shown in FIG. 1 and the cosmetic composition of Embodiment 6 shown in FIG. 2 are indicated by the percentage of each component constituting each cosmetic composition in terms of % by mass. The mass of an actual cosmetic composition is not limited by Examples 1-1 to 6-4 shown in FIGS. 3 to 8. The cosmetic composition according to the present invention may be arbitrarily formed within a scope that satisfies the conditions shown in FIGS. 1 and 2. Each of FIGS. 1 and 2 shows examples of a mode of the cosmetic composition according to the present invention. The percentage of each component may be appropriately changed within a range from the upper limit value to the lower limit value of the percentage of each component shown in FIGS. 1 and 2.

The cosmetic of the present invention can be formed by further adding an additional component to any of the cosmetic compositions having the component ratios according to the formulations of the invention shown in Embodiments 1 to 6 mentioned above.

Specifically, a cosmetic having the efficacy of the cosmetic composition according to Embodiment 1 can be formed by adding one or more members selected from histidine, arginine, proline, isoleucine, serine, valine, aspartic acid, threonine, phenylalanine, pyrrolidonecarboxylic acid, sodium pyrrolidonecarboxylate, sodium lactate, and water to the cosmetic composition of Embodiment 1 mentioned above. Regarding each of these components, each individual component can be added in a quantity corresponding to a component ratio of 0.01% based on the quantity of the cosmetic composition of Embodiment 1, which is regarded as 100%, to thereby produce a suitable cosmetic.

A cosmetic having the efficacy of the cosmetic composition according to Embodiment 2 can be formed by adding one or more members selected from histidine, proline, isoleucine, serine, valine, aspartic acid, threonine, phenylalanine, pyrrolidonecarboxylic acid, sodium pyrrolidonecarboxylate, sodium lactate, and water to the cosmetic composition of Embodiment 2 mentioned above. Regarding each of these components, each individual component can be added in a quantity corresponding to a component ratio of 0.01% based on the quantity of the cosmetic composition of Embodiment 2, which is regarded as 100%, to thereby produce a suitable cosmetic.

A cosmetic having the efficacy of the cosmetic composition according to Embodiment 3 can be formed by adding one or more members selected from histidine, proline, isoleucine, serine, valine, aspartic acid, threonine, phenylalanine, pyrrolidonecarboxylic acid, sodium pyrrolidonecarboxylate, sodium lactate, and water to the cosmetic composition of Embodiment 3 mentioned above. Regarding each of these components, each individual component can be added in a quantity corresponding to a component ratio of 0.01% based on the quantity of the cosmetic composition of Embodiment 3, which is regarded as 100%, to thereby produce a suitable cosmetic.

A cosmetic having the efficacy of the cosmetic composition according to Embodiment 4 can be formed by adding one or more members selected from histidine, proline, isoleucine, serine, valine, aspartic acid, threonine, phenylalanine, pyrrolidonecarboxylic acid, sodium pyrrolidonecarboxylate, sodium lactate, and water to the cosmetic composition of Embodiment 4 mentioned above. Regarding each of these components, each individual component can be added in a quantity corresponding to a component ratio of 0.01% based on the quantity of the cosmetic composition of Embodiment 4, which is regarded as 100%, to thereby produce a suitable cosmetic.

A cosmetic having the efficacy of the cosmetic composition according to Embodiment 5 can be formed by adding one or more members selected from histidine, proline, isoleucine, serine, valine, aspartic acid, threonine, phenylalanine, pyrrolidonecarboxylic acid, sodium pyrrolidonecarboxylate, sodium lactate, and water to the cosmetic composition of Embodiment 5 mentioned above. Regarding each of these components, each individual component can be added in a quantity corresponding to a component ratio of 0.01% based on the quantity of the cosmetic composition of Embodiment 5, which is regarded as 100%, to thereby produce a suitable cosmetic.

A cosmetic having the efficacy of the cosmetic composition according to Embodiment 6 can be formed by adding one or more members selected from arginine, alanine, histidine, proline, isoleucine, serine, valine, aspartic acid, threonine, phenylalanine, pyrrolidonecarboxylic acid, sodium pyrrolidonecarboxylate, sodium lactate, and water to the cosmetic composition of Embodiment 6 mentioned above. Regarding each of these components, each individual component can be added in a quantity corresponding to a component ratio of 0.01% based on the quantity of the cosmetic composition of Embodiment 6, which is regarded as 100%, to thereby produce a suitable cosmetic.

It is obvious from the description that modes with such changes or modifications may be included in the technical scope of the present invention.

The cosmetic composition of the present invention exerts efficacy when applied to hair, the skin, hair of the head and/or the scalp. Use of the method for applying a cosmetic composition according to the present invention for hair, the skin, hair of the head and/or the scalp is useful in fields, for example, hair dressing, beauty, and cosmetics.

What is claimed is:

1. A cosmetic composition comprising:
nucleic acid;
succinic acid;
alanine;
citric acid; and
glycine, wherein
an amount of the nucleic acid is 6.6 to 11.5% by mass based on a total mass of the composition, and
an amount of the citric acid is 7.1 to 17.4% by mass based on the total mass of the composition.

2. The cosmetic composition according to claim 1, wherein
an amount of the succinic acid is 3.6 to 11.5% by mass based on the total mass of the composition,
an amount of the alanine is 3.6 to 11.5% by mass based on the total mass of the composition, and
an amount of the glycine is 3.6 to 23.0% by mass based on the total mass of the composition.

3. The cosmetic composition according to claim 1, further comprising tartaric acid, wherein
the amount of the nucleic acid is 6.6 to 6.7% by mass based on the total mass of the composition,
an amount of the succinic acid is 6.6 to 6.7% by mass based on the total mass of the composition,
an amount of the alanine is 6.6 to 6.7% by mass based on the total mass of the composition,
the amount of the citric acid is 10.0% by mass based on the total mass of the composition,
an amount of the glycine is 13.3 to 13.4% by mass based on the total mass of the composition, and
an amount of the tartaric acid is 56.6 to 56.8% by mass based on the total mass of the composition.

4. The cosmetic composition according to claim 1, further comprising tartaric acid and arginine, wherein
the amount of the nucleic acid is 10.0 to 11.5% by mass based on the total mass of the composition,
an amount of the succinic acid is 10.0 to 11.5% by mass based on the total mass of the composition,
an amount of the alanine is 10.0 to 11.5% by mass based on the total mass of the composition,
the amount of the citric acid is 15.0 to 17.4% by mass based on the total mass of the composition,
an amount of the glycine is 20.0 to 23.0% by mass based on the total mass of the composition,
an amount of the tartaric acid is 2.5 to 30.0% by mass based on the total mass of the composition, and
an amount of the arginine is 5.0 to 23.0% by mass based on the total mass of the composition.

5. The cosmetic composition according to claim 4, wherein
the amount of the nucleic acid is 10.0 to 10.3% by mass based on the total mass of the composition,
the amount of the succinic acid is 10.0 to 10.3% by mass based on the total mass of the composition,
the amount of the alanine is 10.0 to 10.3% by mass based on the total mass of the composition,
the amount of the citric acid is 15.0 to 15.4% by mass based on the total mass of the composition,
the amount of the glycine is 20.0 to 20.6% by mass based on the total mass of the composition,
the amount of the tartaric acid is 28.1 to 30.0% by mass based on the total mass of the composition, and
the amount of the arginine is 5.0 to 5.2% by mass based on the total mass of the composition.

6. The cosmetic composition according to claim 4, wherein
the amount of the nucleic acid is 11.4 to 11.5% by mass based on the total mass of the composition,
the amount of the succinic acid is 11.4 to 11.5% by mass based on the total mass of the composition,
the amount of the alanine is 11.4 to 11.5% by mass based on the total mass of the composition,
the amount of the citric acid is 17.2 to 17.4% by mass based on the total mass of the composition,
the amount of the glycine is 23.0% by mass based on the total mass of the composition,
the amount of the tartaric acid is 10.3 to 10.4% by mass based on the total mass of the composition, and
the amount of the arginine is 14.9 to 15.0% by mass based on the total mass of the composition.

7. The cosmetic composition according to claim 4, wherein
the amount of the nucleic acid is 11.3 to 11.5% by mass based on the total mass of the composition,
the amount of the succinic acid is 11.3 to 11.5% by mass based on the total mass of the composition,
the amount of the alanine is 11.3 to 11.5% by mass based on the total mass of the composition,
the amount of the citric acid is 17.0 to 17.2% by mass based on the total mass of the composition,
the amount of the glycine is 22.6 to 23.0% by mass based on the total mass of the composition,
the amount of the tartaric acid is 2.5 to 3.4% by mass based on the total mass of the composition, and
the amount of the arginine is 22.7 to 23.0% by mass based on the total mass of the composition.

8. The cosmetic composition according to claim 1, further comprising arginine and trehalose, wherein
the amount of the nucleic acid is 7.0 to 7.2% by mass based on the total mass of the composition,
an amount of the succinic acid is 3.6% by mass based on the total mass of the composition,
an amount of the alanine is 3.6% by mass based on the total mass of the composition,
the amount of the citric acid is 7.1 to 7.2% by mass based on the total mass of the composition,
an amount of the glycine is 3.6% by mass based on the total mass of the composition,
an amount of the arginine is 71.4 to 71.5% by mass based on the total mass of the composition, and
an amount of the trehalose is 3.6% by mass based on the total mass of the composition.

9. A cosmetic composition comprising:
glycine; and
trehalose, wherein
an amount of the glycine is 50.0% by mass based on a total mass of the composition, and
an amount of the trehalose is 50.0% by mass based on the total mass of the composition.

10. The cosmetic according to claim 3, further comprising at least one member selected from the group consisting of histidine, arginine, proline, isoleucine, serine, valine, aspartic acid, threonine, phenylalanine, pyrrolidonecarboxylic acid, sodium pyrrolidonecarboxylate, sodium lactate, and water.

11. The cosmetic according to claim 5, further comprising at least one member selected from the group consisting of histidine, proline, isoleucine, serine, valine, aspartic acid, threonine, phenylalanine, pyrrolidonecarboxylic acid, sodium pyrrolidonecarboxylate, sodium lactate, and water.

12. The cosmetic according to claim 6, further comprising at least one member selected from the group consisting of histidine, proline, isoleucine, serine, valine, aspartic acid, threonine, phenylalanine, pyrrolidonecarboxylic acid, sodium pyrrolidonecarboxylate, sodium lactate, and water.

13. The cosmetic according to claim 7, further comprising at least one member selected from the group consisting of histidine, proline, isoleucine, serine, valine, aspartic acid, threonine, phenylalanine, pyrrolidonecarboxylic acid, sodium pyrrolidonecarboxylate, sodium lactate, and water.

14. The cosmetic according to claim 8, further comprising at least one member selected from the group consisting of histidine, proline, isoleucine, serine, valine, aspartic acid, threonine, phenylalanine, pyrrolidonecarboxylic acid, sodium pyrrolidonecarboxylate, sodium lactate, and water.

15. The cosmetic according to claim 9, further comprising at least one member selected from the group consisting of arginine, alanine, histidine, proline, isoleucine, serine, valine, aspartic acid, threonine, phenylalanine, pyrrolidonecarboxylic acid, sodium pyrrolidonecarboxylate, sodium lactate, and water.

16. A method for applying a cosmetic composition to hair, the skin, hair of the head and/or the scalp, comprising:
applying a cosmetic composition according to claim 1 to hair, the skin, hair of the head and/or the scalp.

17. A method for applying a cosmetic composition to hair, the skin, hair of the head and/or the scalp, comprising:
applying a cosmetic composition according to claim 9 to hair, the skin, hair of the head and/or the scalp.
